(19) United States
(12) Patent Application Publication
(10) Pub. No.: US 2010/0040615 A1
(43) Pub. Date: Feb. 18, 2010

(54) Digalactolipidic Antigen Exposed on the Surface of Apicomplex Parasites, and Diagnostic and Therapeutic Use Thereof

(75) Inventors: Cyrille Botte, Grenoble (FR); Nadia Saidani, Montpellier (FR); Maryse Block, Claix (FR); Jean-Francois Dubremetz, Montpellier (FR); Henri Vial, Montpellier (FR); Marie-France Cesbron-Delauw, La Tronche (FR); Corinne Mercier, La Tronche (FR); Eric Marechal, Grenoble (FR)

Correspondence Address: MORGAN LEWIS & BOCKIUS LLP, 1111 PENNSYLVANIA AVENUE NW, WASHINGTON, DC, 20004, US

(73) Assignees: COMMISSARIAT A L'ENERGIE ATOMIQUE, Paris (FR); CENTRE NATIONAL DE LA RECHERCHE SEIENTIFIQUE, Paris (FR)

(21) Appl. No.: 12/526,389

(22) PCT Filed: Feb. 7, 2008
(86) PCT No.: PCT/FR2008/000149

§ 371 Date: Oct. 19, 2009

(30) Foreign Application Priority Data
Feb. 7, 2007 (FR) 0700859

(51) Int. Cl. A61K 39/002; A61K 39/395; A61K 39/385; A61K 39/018; A61K 39/015; A61K 39/012; G01N 33/53; C12N 1/10; C07K 16/00

(52) U.S. Cl. 424/133.1; 424/130.1; 424/151.1; 424/178.1; 424/193.1; 424/269.1; 424/270.1; 424/271.1; 424/272.1; 424/273.1; 435/7.2; 435/258.1; 530/389.1

(57) ABSTRACT

The invention relates to a digalactolipidic antigen exposed on the surface of apicomplex parasites, in the form of a vegetable-type digalactoglycerolipid, and adapted for inducing the production of specific antibodies capable of inhibiting the proliferation and/or the invasive properties of said parasites; the invention also relates to a derived antibody or functional antibody fragment, and to their diagnostic, immunotherapeutic and vaccine applications in human beings or animals.

The present invention relates to a digalactolipid antigen exposed on the surface of apicomplexan parasites in the form of a plant-type digalactoglycerolipid and capable of inducing the production of specific antibodies capable of inhibiting the proliferation and/or the invasive properties of these parasites.

The phylum Apicomplexa groups together several thousand unicellular parasites, among which are major pathologic agents in humans or animals:
the agents for malaria in humans (genus Plasmodium): Plasmodium falciparum, Plasmodium vivax, Plasmodium ovale and Plasmodium malariae,
the agents for piroplasmosis (genus Babesia) in humans (Babesia microti), and animals, in particular B. divergens, B. bigemina, B. major and B. bovis (cattle), B. canis and B. gipsoni (dogs), B. equi and B. caballi (horses),
the agent for human toxoplasmosis (Toxoplasma gondii),
the agent for neosporosis in puppies and other animal species, in particular cattle (Neospora caninum),
the agents for cryptosporidiosis (genus Cryptosporidium), in humans (Cryptosporidium hominis) and animals (C. parvum, for example),
the agents for theileriosis (genus Theileria), in animals (cattle, members of the sheep family, members of the goat family), in particular Theileria annulata and T. parva, and
the agents for other infectious diseases in vertebrates and invertebrates, for example: Sarcocystis neurona (horses), Eimeria tenella (chickens) and Gregarina niphandrodes (invertebrates).

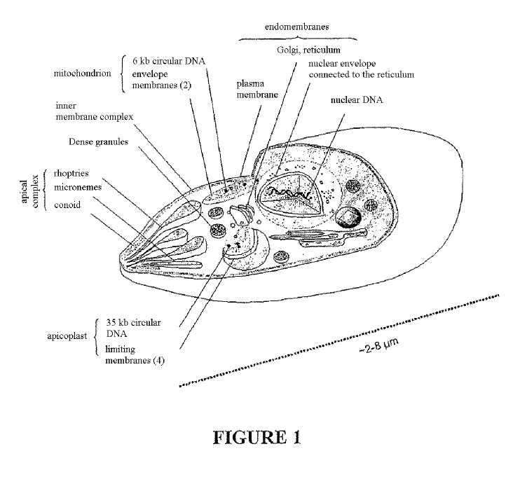

All the parasites of the phylum Apicomplexa (apicomplexan parasites or apicomplexa) share a polarized cellular organization plan, with a basal pole and an apical pole. The membrane compartmentation exhibits particularities compared with a eukaryotic cell of yeast or mammalian type (FIG. 1). Firstly, the plasma membrane is associated with a double membrane, known as the inner membrane complex (IMC). The assembly formed by the inner membrane complex and the plasma membrane constitutes the pellicle. Secondly, in the apical part, the cell exhibits unique secretory organs (rhoptries, micronemes and dense granules), secreted during the invasion of a host cell. Finally, a typical plant organelle, a nonphotosynthetic residual chloroplast called apicoplast, is found in the cell (McFadden et al., Nature, 1996, 381, 482; Köhler et al., Science, 1997, 275, 1485-1489; reviewed in Maréchal E. and M. F. Cesbron-Delauw, Trends Plant Sci., 2001, 6, 200-205).

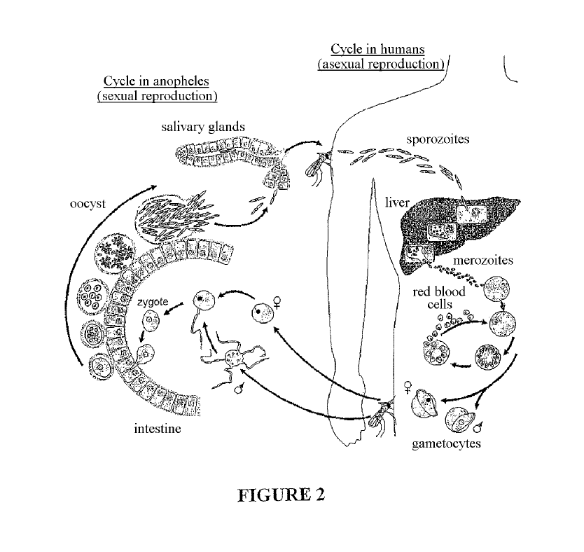

These parasites are transmitted by various vectors. For example, the malaria vector is the female Anopheles mosquito (FIG. 2). Plasmodium enters the body via the blood, in which it develops in a free form called a sporozoite. Later in the cycle of the parasite, Plasmodium goes through an intracellular form in the hepatocytes and then through a multiplication form in the red blood cells. When it passes from one red blood cell to another, a transition which occurs approximately every 48 hours, at the time of the malaria fever attacks, Plasmodium develops in another free blood form called a merozoite.

The development of a subunit vaccine and of an immunodetection test which are effective against infections with apicomplexan parasites, and in particular infections with all the Plasmodium responsible for malaria, has proved to be extremely difficult due to the presence of numerous antigens, to the specificity of these antigens for a particular species of parasite and a particular stage of the parasite cycle, and also to the existence of mechanisms by which the parasites evade the immune response of the human host.

For example, the main rapid immunodetection tests for malaria are based on the detection of surface protein antigens (HRP-2) or secreted protein antigens (soluble antigen; pLDH (plasmodium lactate dehydrogenase) and aldolase), by immunochromatography (reviewed in A. Moody, Clinical Microbiology Reviews, 2002, 15, 66-78).

HRP-2 is an antigen expressed at the surface of erythrocytes and specific for the asexual stages and the young gametocytes of P. falciparum; the HRP-2 immunodetection tests make it possible to detect only infections with P. falciparum, but not infections with Plasmodium virax, Plasmodium ovale and Plasmodium malariae.

Aldolase and pLDH are soluble glycolytic enzymes expressed by the four species of Plasmodium responsible for malaria. However, the sensitivity of the immunodetection tests is weak with respect to Plasmodium ovale and Plasmodium malariae, since the monoclonal antibodies used exhibit a weak affinity for the antigens of these two species of Plasmodium.

An invariant epitope of the surface of the parasitic cells and an antibody directed against this epitope would be advantageous both from the diagnostic point of view and from the prophylactic and therapeutic point of view.

Unlike peptide epitopes which are subjected to rapid genetic variation, the nonpeptide epitopes carried by parasite surface glycoproteins or lipids (glycolipids, glycophospholipids) are more stable and better conserved.

However, among these surface molecules, only some of them are immunogenic in animals. For example, in the case of conventional phospholipids such as phosphatidylcholine and phosphatidylethanolamine it is not possible to awaken the immune system against these compounds which are found in all eukaryotes, and in particular mammals.

Thus, the main nonpeptide surface epitopes of the apicomplexan parasites which have been identified and characterized are carried by glycophospholipids, and in particular the surface glycophosphatidylinositols (GPIs) of Plasmodium. It has in particular been shown that antibodies directed against a conserved region of the carbohydrate groups of the GPIs of P. falciparum neutralize the toxic effects of these GPIs. Thus, immunization with a GPI or a synthetic carbohydrate derived from a GPI of Plasmodium represents a vaccine strategy for preventing the severe forms of malaria (Schofield et al., Nature, 2002, 418, 785-789; Naik et al., Infect. Immun., 2006, 74, 1412-1415). However, this vaccine strategy does not make it possible to prevent infection with Plasmodium.

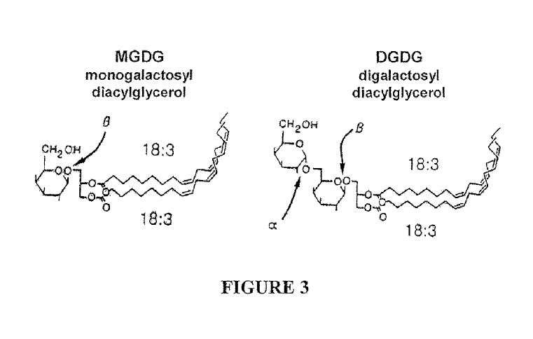

Galactoglycerolipids, and in particular the most abundant ones such as monogalactosyldiacylglycerol (MGDG) and digalactosyldiacylglycerol (DGDG), are lipids specific for plant plasts, which are the cytoplasmic organelles of plant cells that perform photosynthesis. The structure of MGDG and of DGDG comprises a polar head with one (MGDG) or two (DGDG) galactoses, at position 3 of the glycerol (FIG. 3). To date, these two galactoglycerolipids have been detected only in cyanobacteria (the ancestor of plasts) and in eukaryotic cells bearing plasts (plants, algae and protists such as the apicomplexa).

In the apicomplexan parasites, galactoglycerolipids are not very abundant and are undetectable using the conventional methods of lipid extraction and separation by two-dimensional thin layer chromatography (Maréchal et al., Eukaryot. Cell., 2002, 1, 653-656). However, radioactive labeling of the galactoglycerolipids in the presence of UDP-[³H]galactose (Maréchal et al., 2002, mentioned above) or of [¹⁴C]acetate (Bisanz et al., Biochem. J., 2006, 394, 177-205) has made it possible to demonstrate the synthesis of two types of galactoglycerolipids by apicomplexan parasites: a monogalactolipid having a structure compatible with a diradylglycerol, either MGDG or MGAAG (monogalactosylalkylacylglycerol) and a digalactolipid having a structure compatible with a diradylglycerol, either DGDG or DGAAG (digalactosylalkylacylglycerol).

The inventors have shown that the digalactolipid of apicomplexan parasites, and more particularly the digalactose carried by this digalactolipid, is a useful antigen for the diagnosis of and vaccination against infections by all the apicomplexan parasites.

In fact, the anti-digalactolipid antibodies, in particular the anti-DGDG antibodies, specifically recognize all the parasitic stages of the apicomplexa and are therefore useful for the diagnosis of all infections by these parasites, in humans or animals.

Experiments comprising immunolabeling of the apicomplexan parasites (free parasites), without permeabilization of the cell membranes of the parasites, have shown that the localization of the digalactolipid which reacts with the anti-digalactolipid antibody, in particular an anti-DGDG antibody, is not only intracellular (plast), but also at the outer surface of the plasma membrane of the apicomplexan parasites.

This digalactolipid antigen which is present at the outer surface of the parasite can therefore be detected directly at the surface of the free parasites, without a membrane permeabilization step.

Furthermore, the digalactolipid, in particular DGDG, is capable of inducing the production of protective antibodies capable of inhibiting the proliferation and the invasive properties of these parasites. Such antibodies are capable of agglutinating the live parasites and prevent (neutralization) the invasion of host cells by these parasites. They also facilitate phagocytosis of the parasites and activate the classical complement pathway. Consequently, the digalactolipid, in particular DGDG, is a useful antigen for vaccination against infections by all the apicomplexan parasites. Similarly, the antibodies directed against the digalactolipid, in particular DGDG, are useful in passive immunotherapy (serotherapy), against all these infections.

In addition, supplementary experiments have shown that the antigenic fraction of the digalactoglycerolipid of the apicomplexan parasites is constituted of the digalactose part covalently associated with the lipid. In fact, the absence of reactivity of the anti-DGDG sera with MGDG and tri-DGDG indicates that the antibodies directed against DGDG specifically recognize the digalactoside. In addition, immunization with MGDG, under conditions similar to those for DGDG, indicates that MGDG is not capable of inducing a specific antibody response.

Consequently, a subject of the present invention is the use of a digalactosidic antigen covalently associated with a lipid, of an antibody directed against said antigen or of a functional fragment of said antibody, for the preparation of a reagent for use in the diagnosis, or else of a vaccine or of a medicament for use in the prevention or the treatment, of an infection with an apicomplexan parasite in humans or animals.

For the purpose of the present invention, the term “digalactolipid antigen” is intended to mean an antigen comprising a digalactose covalently joined to a lipid or an appropriate molecule derived from a lipid, said antigen being capable of inducing the production of antibodies: (i) directed against this digalactose, (ii) which specifically recognize a digalactolipid of an apicomplexan parasite or apicomplexan parasites, and (iii) capable of inhibiting the proliferation and/or the invasive properties of this or these apicomplexan parasite(s) which is (are) recognized by said antibody.

The inhibition of the proliferation and/or of the invasive properties of the apicomplexan parasites can be measured, in vitro, by means of a conventional test for growth of an apicomplexan parasite or apicomplexan parasites in human or animal cells, in particular by means of a calorimetric test or a fluorescence test, according to the principle described for Toxoplasma gondii (McFadden et al., Antimicrob. Agents Chemother., 1997, 41, 1849-1853; Seebert et al., Gene, 1996, 39-45; Gubbels et al., Antimicrob. Agents Chemother., 2003, 47, 309-316.)

Alternatively, the inhibition of the proliferation and/or of the invasive properties of the apicomplexan parasites can be measured, in vitro, by means of a conventional opsonization test (phagocytosis by macrophages), so as to demonstrate antibodies which increase the phagocytosis of the apicomplexan parasites by macrophages (opsonisant antibodies).

The antigen according to the invention comprises a digalactose covalently associated with a lipid or with an appropriate molecule derived from a lipid, in such a way as to induce the production of specific antibodies. The digalactose may in particular be coupled to a hydrophobic molecule known to those skilled in the art, such as a lipid, or alternatively included in this hydrophobic molecule (glycolipid). The digalactose may also be conjugated to any support known to those skilled in the art as having hydrophobicity properties specific to lipids, according to the conventional techniques of molecular synthesis and coupling.

For the purpose of the present invention, the expression “antibody directed against a digalactosidic antigen” or “anti-digalactose antibody” is intended to mean an antibody produced by immunization of a vertebrate, in particular a mammal, with a digalactolipid antigen.

For the purpose of the present invention, the term “antibody fragment” is intended to mean a functional fragment of said antibody comprising at least the variable domains of the heavy and light chains, such as, in particular, the Fab, Fv and scFv fragments.

For the purpose of the present invention, the term “chimeric antibody”, relative to an antibody of a particular animal species or of a particular antibody class, is intended to mean an antibody comprising all or part of a heavy chain and/or of a light chain of an antibody of another animal species or of another antibody class.

For the purpose of the present invention, the term “humanized antibody” is intended to mean a human immunoglobulin in which the residues of the CDRs (Complementarity-Determining Regions) which form the antigen-binding site are replaced with those of a nonhuman monoclonal antibody having the desired specificity, affinity or activity.

The digalactolipid antigen according to the invention, which is capable of inducing the production of specific antibodies capable of inhibiting the proliferation and/or the invasive properties of apicomplexan parasites, is used as an antigen for vaccination against infections with these parasites.

In addition, this digalactolipid antigen which is capable of being recognized by antibodies specific for apicomplexan parasites is used for the serological diagnosis (indirect diagnosis) of infections with the parasites of the phylum Apicomplexa in humans or animals, in particular for epidemiological studies; the specific antibodies possibly present in the secretions, in particular the serum, of individuals are detected by an appropriate immunochemistry technique, in particular EIA, ELISA, RIA, immunofluorescence, immunoagglutination, immunochromatography, immunocytochemistry, immunohistochemistry, immunoblotting or immunoprecipitation, using a digalactolipid antigen purified according to the conventional techniques known to those skilled in the art.

The antibodies and the antibody fragments as defined above, preferably purified according to the conventional techniques known to those skilled in the art, are used for treating and detecting infections with the parasites of the phylum Apicomplexa (direct diagnosis) in humans or animals.

The detection of the parasites is carried out by means of an appropriate immunochemistry technique, in particular EIA, ELISA, RIA, immunofluorescence, immunoagglutination, immunochromatography, immunocytochemistry, immunohistochemistry, immunoblotting or immunoprecipitation, using a biological sample (blood, serum, faeces, urine, cerebrospinal fluid), taken from an individual who may be infected.

The antibodies directed against the digalactolipid antigen according to the invention, which are capable of inhibiting the proliferation and/or the invasive properties of apicomplexan parasites, are used in serotherapy (passive immunotherapy), for treating infections with these parasites. The human serotherapy is preferably carried out with humanized antibodies, as defined above.

By comparison with nonhuman antibodies, humanized antibodies are less immunogenic and have a sustained half-life in humans because they have only a small proportion of nonhuman sequences, given that virtually all the residues of the FR (framework) regions and of the constant (Fc) region of these antibodies are those of a consensus sequence of human immunoglobulins.

The invention encompasses the use of monoclonal or polyclonal antibodies, of chimeric antibodies such as humanized antibodies, and also of fragments thereof (Fab, Fv, scFv).

The invention encompasses the specific diagnosis of and the specific vaccination against infections with apicomplexan parasites (Apicomplexa) responsible for a pathology in humans or animals (vertebrate or invertebrate), such as, in particular:
the agents for malaria in humans (genus Plasmodium): Plasmodium falciparum, Plasmodium vivax, Plasmodium ovale and Plasmodium malariae,
the agents for piroplasmosis (genus Babesia) in humans (Babesia microti), and animals, for example: B. divergens, B. bigemina, B. major and B. bovis (cattle), B. canis and B. gipsoni (dogs), B. equi and B. caballi (horses),
the agent for human toxoplasmosis (Toxoplasma gondii),
the agent for neosporosis in puppies and other animal species, in particular cattle (Neospora caninum),
the agents for cryptosporidiosis (genus Cryptosporidium), in humans (Cryptosporidium hominis) and animals (C. parvum, for example),
the agents for theileriosis (genus Theileria), in animals (cattle, members of the sheep family, members of the goat family), for example: Theileria annulata and T. parva, and
the agents for other infectious diseases in vertebrates and invertebrates, for example: Sarcocystis neurona (horses), Eimeria tenella (chickens) and Gregarina niphandrodes (invertebrates).

According to one advantageous embodiment of said use, said digalactolipid antigen comprises the digalactose 6-O-(α-D-galactopyranosyl)-β-D-galactopyranose, covalently associated with a lipid.

According to another advantageous embodiment of said use, said digalactolipid antigen is a digalactosyldiradylglyceride of formula I:

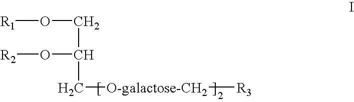

in which R₁ and R₂ each represent, independently of one another, an alkyl or an acyl containing 10 to 25 carbon atoms, and R₃ represents a hydroxyl or sulfonate group.

In accordance with the invention, the R₁ and R₂ residues are identical or different and comprise a substituted or unsubstituted, saturated or unsaturated, linear or branched hydrocarbon-based chain. Preferably, R₁ and R₂ each comprise 0 to 6 carbon-carbon double bonds. R₁ and R₂ are in particular two acyls or one alkyl and one acyl. R₁ and/or R₂ represent(s) in particular the hydrocarbon-based chain of a saturated or unsaturated natural fatty acid. Among the natural fatty acids, mention may in particular be made of saturated acids such as palmitic acid (C₁₅H₃₁—COOH; 16:0) and stearic acid (C₁₇H₃₅—COOH; 18:0), monounsaturated acids such as oleic acid (C₁₇H₃₃—COOH; 18:1), or polyunsaturated acids such as linoleic acid (C₁₇H₃₁—COOH; 18:2) and linolenic acid (C₁₇H₂₉—COOH; 18:3). Preferably, R₁ and/or R₂ represent(s) the carbon-based chain of linolenic acid.

The digalactosyldiradylglyceride of formula I comprises, in particular, the digalactose 6-O-(α-D-galactopyranosyl)-β-D-galactopyranose.

According to an advantageous arrangement of this embodiment, the digalactosyldiradylglyceride of formula I is 1,2-diacyl-(α-D-galactopyranosyl-(1→6)-β-D-galactopyranosyl-(1′→3))-sn-glycerol (DGDG).

A subject of the present invention is also a reagent for the diagnosis of an infection with an apicomplexan parasite, characterized in that it is constituted of a digalactolipid antigen as defined above, an antibody directed against said antigen or a fragment of said antibody, as defined above.

The reagents may be coupled to particles or to an appropriate label. The particles are, in particular, gold particles, liposomes (liposomes containing a dye) or erythrocytes (immunoagglutination). The label is a substance which produces a signal that can be detected by conventional methods or which reacts with another substance, so as to produce a detectable signal. Among the labels that can be used in the diagnostic methods according to the invention, mention may in particular be made of chromophores, fluorescent or chemiluminescent substances, enzymes, radioactive isotopes and complexing agents (protein A, protein G, biotin, lectin).

The reagents as defined above may also be immobilized on an appropriate support, according to the conventional methods known to those skilled in the art.

Among the appropriate supports on which these reagents may be immobilized, mention may in particular be made of those made of plastic (latex, polystyrene, polyvinylchloride, polyurethane, polyacrylamide, polyvinyl acetate), of glass or of paper (nitrocellulose). These supports are in the form of plates (microplates), sheets, strips or particles (beads). The immobilization of the reagents on the support can be carried out by the formation of bridging between the reagent and a molecule which is coupled to the support (hydrazide, protein A, glutaraldehyde, carbodiimide or lysine), according to conventional techniques.

A subject of the present invention is also an in vitro method for detecting an infection with an apicomplexan parasite, using a biological sample, which method is characterized in that it comprises at least:

(a) bringing said biological sample into contact with at least one antibody, one antibody fragment or one digalactolipid antigen, as defined above, and

(b) revealing the antigen-antibody complexes formed in (a), by any appropriate means.

The invention encompasses the conventional immunochemistry methods, for example: EIA, ELISA, RIA, immunofluorescence, immunoagglutination, immunochromatography, immunocytochemistry, immunohistochemistry, immunoblotting and immunoprecipitation.

According to one advantageous embodiment of said method, step (a) comprises bringing said biological sample into contact successively with at least a first capture antibody directed against the digalactolipid antigen and at least a second antibody specific for the parasite to be detected and different than the first antibody, or alternatively with a fragment of the first and/or of the second antibody. The use of a second antibody which is genus-specific (Plasmodium genus, for example) or species-specific (P. falciparum or P. vivax species, for example) advantageously makes it possible to determine the parasite genus or species responsible for the infection.

According to another advantageous embodiment of said method, it comprises, prior to or concomitantly with step (a), a membrane permeabilization step.

The membrane permeabilization of the host cells makes it possible to detect the intracellular stages of the parasite. The membrane permeabilization of the parasites makes it possible to detect the internal antigens (not exposed to the surface of the plasma membrane of the parasite).

The membrane permeabilization is carried out under the standard conditions, according to the conventional methods known to those skilled in the art. It can in particular be carried out in the presence of detergent, such as Triton-X100, at concentrations of the order of 0.1%.

Said method is advantageously an ELISA immunocapture method in which:
step (a) comprises:
(a₁) bringing said biological sample into contact with at least a first antibody or an antibody fragment directed against the digalactolipid antigen as defined above, which is attached to an appropriate support, in particular a microplate,
(a₂) washing the solid phase, and
(a₃) adding at least a second antibody or an antibody fragment, different than the first, said antibody or antibody fragment optionally being appropriately labeled, and
   step (b) of revealing the antigen-antibody complexes formed is carried out either directly using a second antibody which is labeled, for example, with biotin or an appropriate enzyme such as peroxidase or alkaline phosphatase, or indirectly using an anti-immunoglobulin serum which is labeled as above. The complexes thus formed are revealed by means of an appropriate substrate.

For example:
step (a₁) is carried out with at least a first monoclonal or polyclonal antibody or a fragment thereof, directed against the digalactolipid antigen, as defined above, and
step (a₃) is carried out with at least one antibody or one antibody fragment directed against another antigen specific for the parasite of the phylum Apicomplexa to be detected; when an antibody or an antibody fragment directed against an internal antigen is used, said antibody is incubated in the presence of detergent, such as Triton X-100 for example, at concentrations of the order of 0.1%.

Alternatively, said method is an immunochromatography method. In a first step, the parasites possibly present in the biological sample are first of all captured in the liquid phase by a capture antibody or capture antibody fragment (for example, an antibody conjugated to gold particles or to liposomes containing a dye based on selenium), directed against the digalactolipid antigen as defined above. In a second step, the antigen-antibody complexes formed migrate along the nitrocellulose strip and are subsequently captured by a second antibody and optionally a third antibody (in the direction of migration), specific for the parasite to be detected and different than the first antibody, which antibodies are immobilized on a nitrocellulose strip, hence the appearance of a colored line at the level of the second and, optionally, of the third antibody.

The use of two different antibodies immobilized on the nitrocellulose strip, the first having a specificity greater than or equal to the second, makes it possible to type the parasite genus (Plasmodium, for example) and/or species (P. falciparum or P. vivax, for example) which is responsible for the infection. For example, the first antibody and the second antibody are, respectively: a genus-specific antibody and a pan-specific antibody; a species-specific antibody (the P. falciparum species, for example) and a genus-specific antibody (the Plasmodium genus, for example); or two antibodies directed against different species (P. falciparum and P. vivax, for example). In addition, a control antibody, immobilized downstream of the above antibodies, makes it possible to verify that the antibodies present in the liquid phase have indeed migrated along the strip.

A subject of the present invention is also a kit for detecting an infection with a parasite of the phylum Apicomplexa, characterized in that it comprises at least one digalactolipid antigen or at least one antibody or one antibody fragment, as defined above. The kit advantageously comprises at least a second antibody as defined above, preferably a second antibody specific for the genus or for the species of the parasite to be detected.

A subject of the present invention is also an antibody directed against a digalactolipid antigen or a fragment of said antibody, as defined above, for use as a medicament in the prevention or treatment of an infection with a parasite of the phylum Apicomplexa.

A subject of the present invention is also a pharmaceutical composition, characterized in that it comprises at least one antibody directed against a digalactolipid antigen or one fragment of said antibody, as defined above, a pharmaceutically acceptable vehicle and, optionally, a carrier substance.

A subject of the present invention is also an immunogenic or vaccine composition, characterized in that it comprises a digalactolipid antigen as defined above, optionally combined with one or more antigens of interest, in particular antigens specific for apicomplexan parasites, a pharmaceutically acceptable vehicle and, optionally, an adjuvant or a carrier substance.

The compositions according to the invention are in a galenic form suitable for parenteral (subcutaneous, intramuscular, intradermal, intravenous), enteral (oral, sublingual) or local (nasal, rectal, vaginal) administration.

The compositions according to the invention comprise an effective dose of vaccinating antigen or of antibody. The effective dose of antigen is the dose necessary to induce a specific-antibody response capable of preventing, of reducing or of treating the parasitic infection and the associated symptoms. The effective dose of antibody is the dose capable of preventing, of reducing or of treating the parasitic infection and the associated symptoms. The effective dose depends on the type of pathogenic agent, on the route of administration and the frequency of the administrations, on the type of mammal to be treated, and also on other factors, known to those skilled in the art.

The pharmaceutically acceptable vehicles, the carrier substances and the adjuvants are those conventionally used.

The adjuvants are advantageously chosen from the group constituted of: alumina hydroxide, squalene, saponin, oily emulsions, inorganic substances, bacterial extracts (BCG, PPD, toxoid), cytokines, etc.

The carrier substances are advantageously selected from the group constituted of: unilamellar or multilamellar liposomes, ISCOMs, virosomes, viral pseudoparticles, saponin micelles, solid microspheres which are saccharide (poly(lactide-co-glycolide)) or gold-bearing in nature, and nanoparticles.

A subject of the present invention is also a monoclonal or chimeric antibody, or a fragment of said antibody, directed against a digalactosidic antigen as defined above. Preferably, it is a humanized antibody or a fragment of said antibody.

The digalactolipid antigen according to the invention and the antibodies have the following advantages over the antigens/antibodies of the prior art:
the antigen is present at all the parasitic stages, in all the apicomplexan parasites containing plasts. It is detectable on free parasites, without a membrane permeabilization step. The detection of the antigen or of the antibodies directed against this antigen therefore enables the diagnosis of all infections with the apicomplexan parasites, in humans and animals,
the antigen induces immunity against infection by all the apicomplexan parasites. A single vaccine comprising this antigen makes it possible to reduce the incidence of infections with all the apicomplexan parasites and the associated pathologies, in humans and in animals.

In addition, among these antigens, DGDG has the additional advantage of being available in large amounts in plants, and the purification thereof is simple, thereby limiting the production costs for the diagnostic reagents and the vaccines.

These various reagents are prepared and used according to the conventional techniques of biochemistry and immunology, by following the standard protocols such as those described in Current Protocols in Immunology (John E. Cologan, 2000, Wiley and Son Inc. Library of Congress, USA) and in Antibodies: A Laboratory Manual (E. Howell and D. Lane, Cold Spring Harbor Laboratory, 1988).

More specifically:
the DGDG is extracted from plants and then purified according to the conventional methods as described, in particular, in Andersson et al., Journal of Chromatography A, 1997, 785, 337-343; Deschamps et al., Journal of Chromatography A, 2004, 1040, 115-121; Aro et al., Journal of Cereal Science, 2007, 45, 116-119;
the polyclonal antibodies are prepared by immunization of an appropriate animal with an antigen as defined above, optionally conjugated to a carrier protein such as KLH or to albumin and/or combined with an appropriate adjuvant such as Freund's (complete or incomplete) adjuvant or alumina hydroxide; after a satisfactory antibody titer has been obtained, the antibodies are harvested by taking the serum from the immunized animals and enriched in immunoglobulins by precipitation, according to conventional techniques, and then the specific immunoglobulins are optionally purified by appropriate techniques known to those skilled in the art, in particular by chromatography on a column to which protein A or the antigen as defined above is attached, so as to obtain a preparation of monospecific immunoglobulins;
the monoclonal antibodies are produced from hybridomas obtained by fusion of B lymphocytes from an animal immunized with the antigen as defined above, with myelomas, according to the technique of Köhler and Milstein (Nature, 1975, 256, 495-497); the hybridomas are cultivated in vitro, in particular in fermenters, or produced in vivo, in the form of ascites; alternatively, said monoclonal antibodies are produced by genetic engineering as described in American patent U.S. Pat. No. 4,816,567. For example, animals are strongly and repeatedly immunized with the antigen as defined above, according to a standard protocol comprising a first immunization by multisite subcutaneous injection of the antigen in an equivalent volume of complete Freund's adjuvant, and then 10 days later, a series of four boost immunizations 2 weeks apart, this time with incomplete Freund's adjuvant; the first two immunizations are carried out by subcutaneous and intramuscular injection and the last two by intramuscular and intravenous injection. The monoclonal antibodies are produced according to a standard protocol which comprises sacrificing the animals two weeks after the final booster, removing the spleen, placing the splenic lymphocytes in suspension and fusing these lymphocytes with the SP2/0 cell line (this murine line does not produce any murine antibody, is immortalized, and has all the secretion machinery necessary for the secretion of immunoglobulins);
the antibody fragments are produced from cloned V_{H} and V_{L} regions, or from the mRNAs of hybridomas or of splenic lymphocytes of an animal immunized with the antigen as defined above; for example, the Fv, scFv or Fab fragments are expressed at the surface of filamentous phages according to the technique of Winter and Milstein (Nature, 1991, 349, 293-299); after several selection steps, the antigen-specific antibody fragments are isolated and the cDNAs corresponding to said fragments are expressed in an appropriate expression system, using the conventional techniques for cloning and expression of recombinant DNA;
the humanized antibodies are produced by general methods such as those described in international application WO 98/45332.

The monoclonal and polyclonal antibodies or fragments thereof as defined above, are purified by conventional techniques known to those skilled in the art, in particular by chromatography on a column to which protein A or the digalactolipid antibody as defined above is attached.

In addition to the above arrangements, the invention also comprises other arrangements, which will emerge from the description that follows, which refers to exemplary embodiments of the subject of the present invention, with reference to the attached drawings in which:

FIG. 1 is a diagram of the organization of an apicomplexan cell;

FIG. 2 illustrates the cycle of Plasmodium falciparum, the agent for malaria;

FIG. 3 illustrates the structure of monogalactosyldiacylglycerol (MGDG) and of digalactosyldiacylglycerol (DGDG), two major galactolipids of the plasts of plants and of algae, and of the membranes of cyanobacteria. The fatty acids esterified at positions 1 and 2 of the glycerol are examples: the carbon-chain lengths and the number and position of double bonds, and also the possible presence of oxidation, can vary;

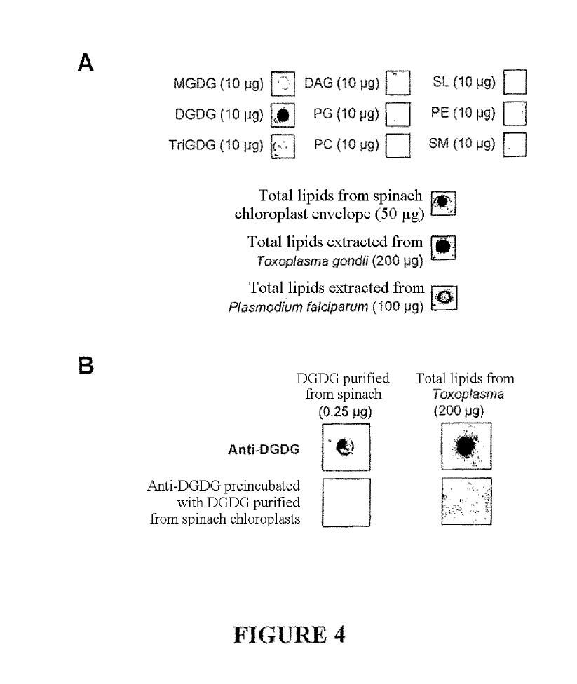

FIG. 4 illustrates the specificity of a serum obtained by immunization of a rabbit with DGDG purified from spinach chloroplasts.

A. The serum diluted to 1/100th was tested on a series of pure lipids and of total lipids from spinach chloroplast envelope, and also of total lipids extracted from Toxoplasma gondii and from Plasmodium falciparum, deposited on a nitrocellulose membrane. MGDG: monogalactosyldiacylglycerol. DGDG: digalactosyl-diacylglycerol. TriGDG: trigalactosyldiacylglycerol. DAG: diacylglycerol. PG: phosphatidylglycerol. PC: phosphatidylcholine. SL: sulfolipid. PE: phosphatidylethanolamine. SM: sphingomyelin.

B. The reactivity of the antibody is abolished after preincubination in the presence of DGDG purified from spinach chloroplasts;

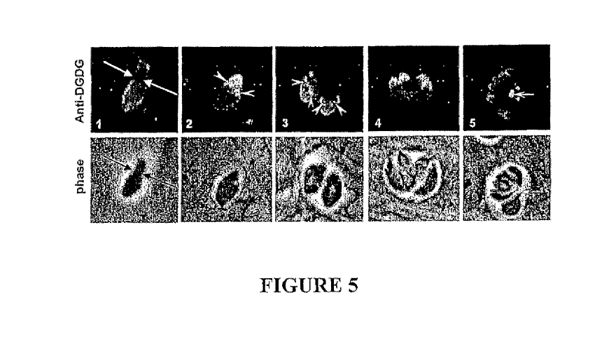

FIG. 5 illustrates the immunolabeling of Toxoplasma gondii inside human fibroblasts, using an anti-DGDG serum.

The upper panels (1 to 5) illustrate the analysis of the labeling with the anti-DGDG serum, by fluorescence microscopy. The lower panels represent the visualization of the cells by phase microscopy. 1. Penetration of a free Toxoplasma into a human cell (at the level of the point of constriction indicated by arrows). 2. A Toxoplasma inside a human fibroblast, preparing its division. The apex is rich in epitopes recognized by the anti-DGDG serum. The two daughter cells are indicated by arrows and each have a galactolipid stop detected in the apical position. 3. Two Toxoplasma inside a human fibroblast, preparing their division. 4. Four Toxoplasma inside a human fibroblast. 5. More than eight Toxoplasma inside a human fibroblast;

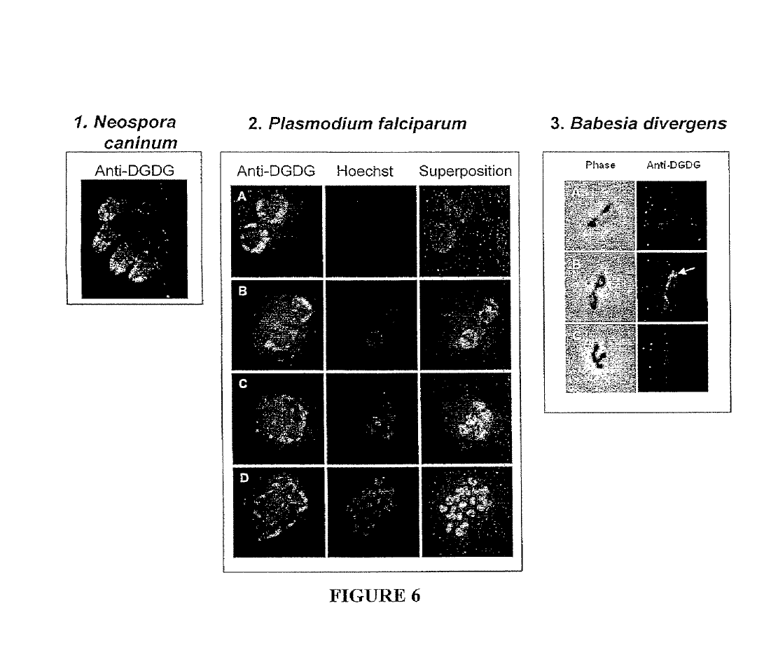

FIG. 6 illustrates the labeling of Neospora caninum (1), Plasmodium falciparum (2) and Babesia divergens (3) cells using a serum from a rabbit immunized with DGDG purified from spinach chloroplasts (Anti-DGDG). The three immunolabelings are shown for intracellular stages of the parasites. The labeling by the Hoechst method shown in the case of Plasmodium makes it possible to pinpoint the nuclei of the parasitic cells. The cells infected with Babesia divergens are visualized, in parallel, by phase microscopy;

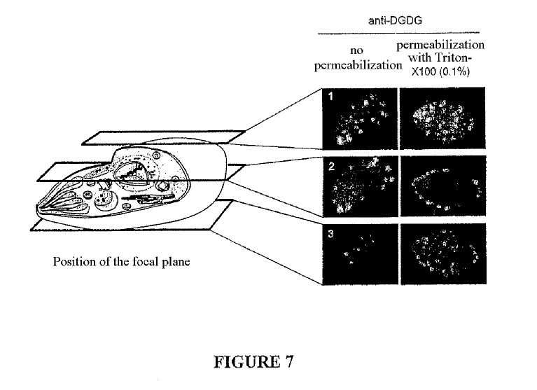

FIG. 7 illustrates the immunolabeling of the free form of Toxoplasma gondii using a serum from a rabbit immunized with DGDG purified from spinach chloroplasts. The immunolabelings were carried out using an anti-DGDG serum diluted to 1/25th, with permeabilization using the detergent Triton X-100 (images 1-3 on the right) or without permeabilization (images 1-3 on the left);

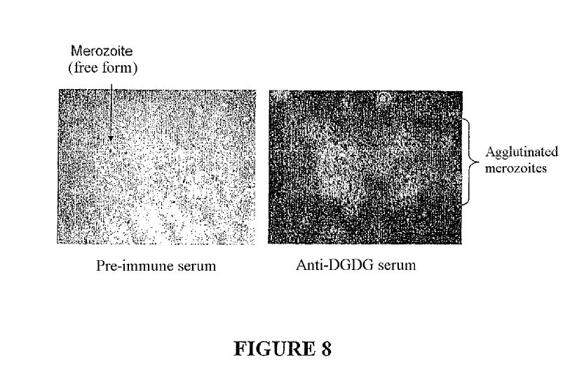

FIG. 8 illustrates the immunoagglutination of merozoites (free blood forms) of Plasmodium falciparum using a serum from a rabbit immunized with DGDG purified from spinach chloroplasts (anti-DGDG serum) The preimmune serum shows no agglutinating property;

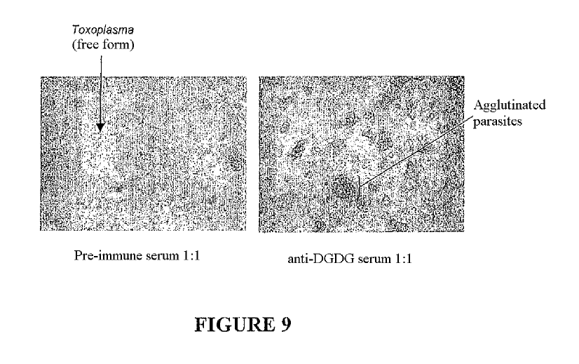

FIG. 9 illustrates the immunoagglutination of free forms of Toxoplasma gondii using a serum from a rabbit immunized with DGDG purified from spinach chloroplasts (anti-DGDG serum). The preimmune serum shows no agglutinating property;

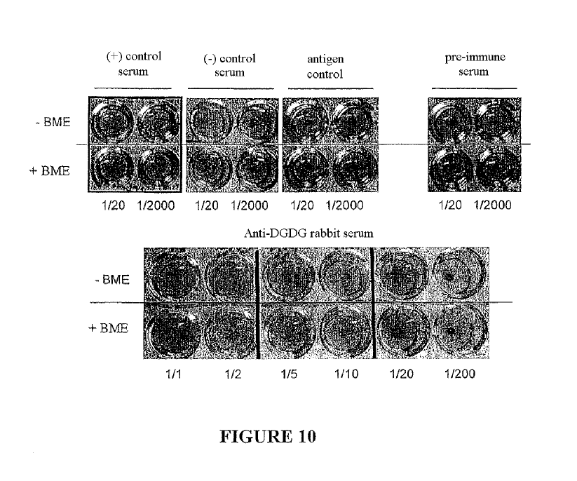

FIG. 10 illustrates the immunoagglutination of Toxoplasma using the Toxo-Screen DA® kit from Biomerieux. The upper part shows the result of this test for control sera, either which do not immunoagglutinate the toxoplasms ((−) control serum; sedimentation of the toxoplasms in a spot or in a ring), or which agglutinate the toxoplasms in the form of a cloud ((+) control serum; framed wells). The lower part shows a test carried out in parallel using various dilutions of rabbit sera (α-DGDG); the cloud corresponding to an immunoagglutination is observed starting from the 1/2 dilution of the anti-DGDG serum;

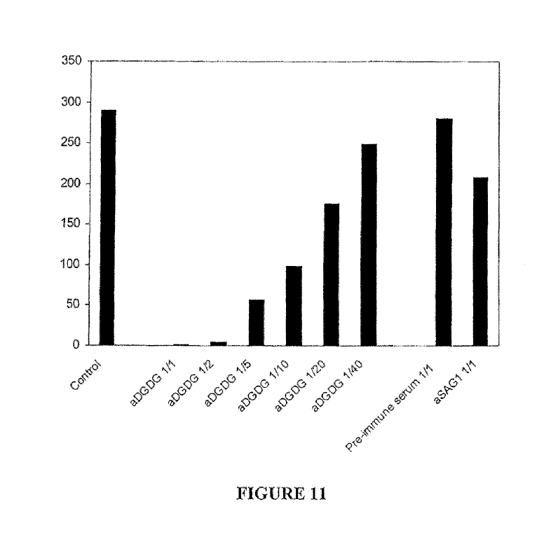

FIG. 11 illustrates the effect of the anti-DGDG antibody (aDGDG; dilutions 1/40 to 1/1) on the invasion of human cells by Toxoplasma gondii. The cells incubated with Toxoplasma gondii, alone (control) or in the presence of a pre-immune serum or of antibodies directed against the major surface antigen of Toxoplasma gondii, SAG1 or p30 (aSAG1), serve as controls;

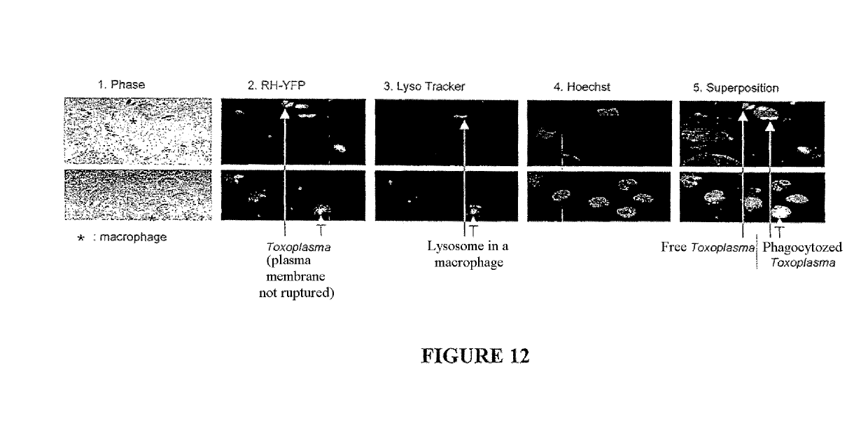

FIG. 12 illustrates the principle of the Toxoplasma gondii cell opsonization test. Toxoplasms expressing the yellow fluorescent protein YFP (RH-YFP recombinant line) are incubated with rat macrophages and the phagocytosis of the toxoplasm cells by the macrophages is observed.

1. Phase microscopy.

2 to 5 fluorescence microscopy: 2. Visualization of the emission of fluorescence (540 nm) by the recombinant toxoplasms (RH-YFP) after excitation at 488 nm. 3. Labeling of the macrophage lysosomes generated by the phagocytosis, using a red fluorescent probe (Lyso-Tracker red, Invitrogen). 4. Visualization of the macrophage nuclei by Hoechst staining. 5. Superimposition of the YFP protein fluorescence (green fluorescence) and of the Lyso-Tracker fluorescence (red fluorescence) makes it possible to detect the parasites phagocytosed by the macrophages (yellow fluorescence);

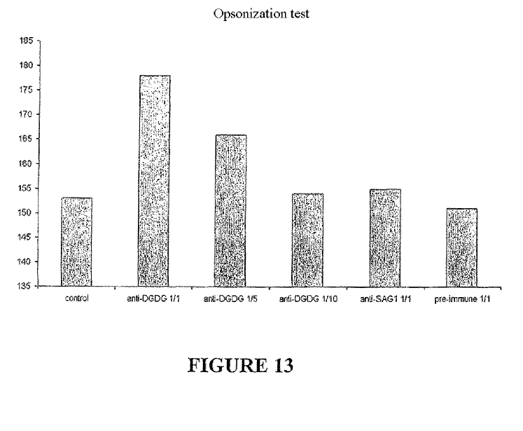

FIG. 13 illustrates the effect of the anti-DGDG antibody (aDGDG; dilutions 1/10, 1/5 and 1/1) on Toxoplasma gondii cell opsonization. The macrophages incubated with Toxoplasma gondii alone (control), or Toxoplasma gondii preincubated with a pre-immune serum or antibodies directed against the major surface antigen of Toxoplasma gondii, SAG1 or p30 (aSAG1), serve as controls;

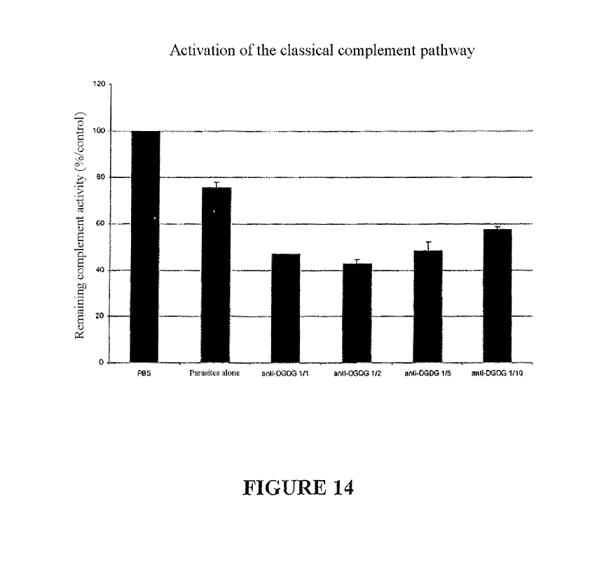

FIG. 14 illustrates the effect of the anti-DGDG antibodies incubated in the presence of T. gondii on the classical complement activation pathway by measuring the time necessary to lyse 50% of the sensitized erythrocytes (TH50). The parasites (8×10⁶ per condition) were preincubated in the presence of various dilutions of an anti-DGDG rabbit serum. After removal of the pre-treatment medium, the toxoplasms are placed in solution with human serum seronegative for toxoplasmosis (NHS, containing the complement proteins) and then with sensitized red blood cells. The effectiveness of each serum dilution in terms of activating the classical complement pathway is assessed by the time necessary to lyse 50% of the sensitized red blood cells and by comparison of this time with the time measured for lysing 50% of the red blood cells incubated in the presence of PBS and of NHS. The remaining complement activity is expressed by the ratio: TH50 control (PBS+NHS+red blood cells)/TH50 of the sample (parasites+serum+NHS+red blood cells)×100. The results correspond to the measurement carried out during three independent experiments in triplicate.

### EXAMPLE 1

### Production of Antibodies Directed Against DGDG

### 1) Materials and Methods

### a) Extraction and Purification of DGDG

The DGDG is purified from extracts of spinach chloroplasts, according to the protocol described in Williams et al., J. Lipid Research, 1975, 16, 61-66 or Jouhet et al., FEBS Letters, 2003, 544, 63-68.

b) Immunization of Animals with DGDG

Two rabbits and one rat were immunized with purified DGDG (2.5 mg per immunization), according to the following protocol:
a first immunization (D₀) by multisite subcutaneous injection (total volume of 1 ml) of 0.5 mg of antigen in 0.5 ml of a solution of NaCl (0.9%), emulsified in 0.5 ml of complete Freund's adjuvant,
a second immunization (D₁₀) by subcutaneous (1 ml) and intramuscular (0.5 ml) injection of 0.5 mg of antigen in 0.75 ml of a solution of NaCl (0.9%), emulsified in 0.75 ml of incomplete Freund's adjuvant,
a third immunization (D₂₁) by subcutaneous (1 ml) and intramuscular (0.5 ml) injection of 0.5 mg of antigen in 0.75 ml of a solution of NaCl (0.9%), emulsified in 0.75 ml of incomplete Freund's adjuvant,
a fourth immunization (D₃₆) by intramuscular (0.5 ml) and intravenous (0.5 ml) injection: 0.17 mg of antigen in 0.25 ml of a solution of NaCl (0.9%), emulsified in 0.25 ml of incomplete Freund's adjuvant, injected intramuscularly, and 0.33 mg of antigen in 0.5 ml of a solution of NaCl (0.9%), injected intravenously, and
a fifth immunization (D₅₀) by intramuscular (0.5 ml) and intravenous (0.5 ml) injection: 0.17 mg of antigen in 0.25 ml of a solution of NaCl (0.9%), emulsified in 0.25 ml of incomplete Freund's adjuvant, injected intramuscularly, and 0.33 mg of antigen in 0.5 ml of a solution of NaCl (0.9%), injected intravenously.

The sera were taken at D₅₇; the sera taken before the immunization (pre-immune serum) serve as control.

### c) Immunoblotting

The lipids (lipid extracts or purified lipids) diluted in chloroform or butanol are deposited at the surface of a nitrocellulose membrane, and incubated in the presence of the sera diluted to 1/100th, and the antigen-antibody complexes formed are revealed using a secondary antibody coupled to peroxidase and a chemoluminescent substrate (ECL, Amersham), according to the conventional immunoblotting protocols.

### 2) Results

The immunization of the animals with the plant digalactolipid, DGDG, made it possible to obtain active sera containing antibodies that recognize DGDG (FIG. 4). The immunization of a rabbit with the other major plastid galactolipid of plants, MGDG, according to the same protocol, did not make it possible to obtain active serum.

### EXAMPLE 2

### Analysis of the Specificity of the Anti-DGDG Antibodies

### 1) Materials and Methods

The activity of the sera directed against the plastid digalactolipid (DGDG) is analyzed by immunoblotting, according to the protocol described in example 1.

### 2) Results

All the immune sera have a DGDG-binding activity (FIG. 4A). The sera recognize the DGDG from plants (spinach extracts) and from apicomplexan parasites (Toxoplasma and Plasmodium; FIG. 4A), whereas the pre-immune sera show no DGDG-binding activity.

The binding activity of the immune sera is DGDG-specific; no binding is detected with other lipids (FIG. 4A) and the binding is abolished when the sera are preincubated in the presence of DGDG (FIG. 4B).

In addition, the absence of reactivity of the anti-DGDG sera with MGDG and tri-DGD indicates that the antibodies directed against DGDG specifically recognize the digalactoside part covalently associated with the glycerolipid part.

### EXAMPLE 3

### Detection and Localization of DGDG in the Cell Membranes of Apicomplexan Parasites

### 1) Materials and Methods

The apicomplexan parasites, in free or intracellular form, are immunolabeled with the anti-DGDG sera of example 1 (directly or after membrane permeabilization with a low concentration of Triton-X100) and/or labeled with the Hoechst dye which stains the nuclei. The labeling of the free or intracellular parasites is analyzed by fluorescence microscopy.

2) Results

The immunolabeling of apicomplexan parasites using the antibodies directed against the plastid digalactolipid (anti-DGDG antibody of example 1) and the detection by fluorescence microscopy show that the apicomplexan parasite cells are immunolabeled after membrane permeabilization using a low concentration of the detergent Triton-X100 (FIG. 5). The DGDG is always detected in the parasite cells and is never detected on the human cell. These labelings show that the digalactolipid of the parasites is localized in a structure which opens out before cell division (FIG. 5, panels 2 and 3). This labeling is typical of the inner membrane complex, the fission of which precedes cell division. The apicomplexan parasite labeling is observed on all the parasitic stages, in Toxoplasma (FIG. 5), Plasmodium, Neospora and Babesia (FIG. 6).

The immunolabeling of the free parasitic cells, with or without membrane permeation using the detergent Triton-X100 (FIG. 7), shows, in both cases, the labeling at the periphery of the cell, following the outline of the cell, when the focal plane of the observation is set from the closest part to the furthest part of the cell (see explanatory diagram of FIG. 7). These results indicate that the epitope recognized by the anti-DGDG antibody is localized, in addition to the inner membrane complex, at the outer surface of the plasma membrane of the apicomplexan parasites.

### EXAMPLE 4

### Immunoagglutination of Apicomplexan Parasites Using Anti-DGDG Serum Obtained after Immunization with Plant Digalactolipid

### 1) Materials and Methods

### a) Agglutination of Live Apicomplexan Parasites

Live parasites are incubated with various dilutions (1:10 to 1:1 in PBS) of the anti-DGDG serum or of the pre-immune serum of example 1, inactivated beforehand for 30 mins at 37° C., and the parasites are observed by optical microscopy.

### b) Direct Agglutination of Sensitized Apicomplexan Antigens

The Toxoplasma gondii immunoagglutination test is described in Desmonts, G. and J. S. Remington, J. Clin. Microbiol., 1980, 11: 562-568. This test is the reference test for detecting anti-toxoplasm IgGs (Toxo-Screen DA® kit, reference 75 481, Biomerieux).

The antigen used is a suspension of formalized toxoplasms (Sabin strain), diluted in an albumin buffer, pH 8.95. The kit comprises a positive goat serum, calibrated relative to the WHO standard (positive CTR) and a negative goat serum (negative CTR). The agglutination is carried out in round-bottom microtitration plates. The test is carried out in duplicate for each serum: absence of reducing agent (2-beta-mercaptoethanol, 2-ME or B-ME) along the first row (−B-ME); presence of 2-ME along the second row (+B-ME). The reducing agent makes it possible to denature the IgMs and therefore to search for their presence in the serum.

The sera are diluted in PBS and distributed into the wells, 2-ME (25 μl; 2 mol/l) is added to the wells of the second row, and then the suspension of antigen diluted to 1/5 (50 μl) is added to all the wells. After homogenization, the plate is covered with a self-adhesive sheet and incubated for 5 to 18 hours at ambient temperature with no vibration.

The plates are read in the following way (FIG. 10):
antigen control: sedimentation of the toxoplasms as a spot or as a ring,
positive reaction: agglutination of the toxoplasms in the form of a cloud,
negative reaction: sedimentation of the toxoplasms in a spot or in a ring, and
limiting reaction: agglutination in the form of a cloud covering half the bottom of the well.

### 2) Results

The agglutinating properties of the sera obtained after immunization of rabbits with DGDG purified from plants were tested on live or formalized apicomplexan parasites.

FIG. 8 shows the agglutination of live merozoites (free form) of the agent for malaria (Plasmodium falciparum), with an anti-DGDG serum diluted to ⅕th. FIG. 9 shows the agglutination of free forms of Toxoplasma gondii with an anti-DGDG serum; the immunoagglutination is visible from the 1:10 concentration onward, and is at a maximum at the 1:1 dilution. The pre-immune serum shows no agglutinating property.

FIG. 10 shows the agglutination of formalized toxoplasms, with an anti-DGDG serum diluted to 1/2 and to 1/20. This serum contains IgMs since the B-ME reduces the agglutination by 50%.

### EXAMPLE 5

### The Anti-DGDG Antibodies Inhibit the Infection of Human Cells with Toxoplasma gondii

### 1) Materials and Methods

The infection of human cells with Toxoplasma gondii is analyzed by means of a test for assaying beta-glactosidase in cells of the HFF (Human foreskin fibroblast) line infected with a recombinant toxoplasma strain comprising the LacZ gene under the control of the SAG1 promoter, according to the method described by McFadden, D. C. et al., Antimicrobial agents and chemotherapy, 1997, 41, 1849-1853; Seeber, F. and Boothroyd, J. C., Gene, 1996, 169, 39-45.

The effect of the anti-DGDG antibodies on the infection of the HHF cells with Toxoplasma gondii is tested at dilutions of 1:40 to 1:1. The cells incubated with Toxoplasma gondii, alone (control) or in the presence of a pre-immune serum or else of antibodies directed against the major surface antigen of Toxoplasma gondii, SAG1 or p30 (aSAG1), serve as controls.

### 2) Results

FIG. 11 shows that the anti-DGDG antibodies inhibit the infection of human cells with Toxoplasma gondii; the effect is visible from the 1:20 concentration onward and is at a maximum at the 1:2 dilution. The pre-immune serum has no effect and the anti-SAG1 antibodies have a very weak effect.

### EXAMPLE 6

### The Anti-DGDG Antibodies Increase the Opsonization of Toxoplasma gondii

### 1) Materials and Methods

Recombinant toxoplasmas RH-YFP (Gubbels et al., Antimicrobial agents and Chemotherapy, 47, 309-306) are incubated with various antibodies (anti-DGDG 1:1, 1:5 and 1:10; pre-immune serum (1:1) and anti-SAG1 antibody (1:1)), for 30 min at 37° C., with shaking (orbital wheel), centrifuged for 10 min at 2000 rpm (washing), and then counted.

Rat macrophages are deposited on cover slips (5000 cells/cover slip) coated with polylysine (1 mg/ml), and are incubated for 2 h with Lysotracker Red DND-99 (dilution 1/20 000; Invitrogen). The macrophages are then infected with the toxoplasms (50 toxoplasmas/macrophage), for 2 h to 4 h at 37° C. (phagocytosis/invasion) in the presence of Lysotracker, washed, and then fixed for 20 min with 5% PFA and stained with Hoechst (1/15 000).

The YFP-protein fluorescence and the lysotracker fluorescence are analyzed by confocal microscopy.

2) Results

The in vitro opsonization test shows a 20% stimulation in the presence of anti-DGDG antibodies (FIG. 13).

### EXAMPLE 7

### The Anti-DGDG Antibodies Activate the Classical Complement Pathway by Virtue of the Opsonization of the Parasite

The ability of the anti-DGDG serum (example 1) to activate the classical complement pathway was evaluated. The complement system is a biochemical cascade of the innate immune system which involves 9 main proteins (C1 to C9). Activation of this system enables the destruction of pathogens and the recruitment of a large number of participants in the immune system for a regulated response (for a general review, Janeway et al., Annu Rev Immunol., 2002, 20, 197-216). The conventional pathway is activated by the antigen-antibody complex. Interaction of the C1 protein complex with the constant fragment of two (or more) IgGs (Ig1, Ig2 and Ig3) or else of a pentameric IgM makes it possible to initiate the cascade. The antigen-antibody-C1 complex makes it possible to cleave the C4 and C2 proteins, which associate with one another and cleave the C3 protein so as to give C3a and C3b. C3b fixes to the membrane of the pathogen, leading to the formation, with other complement proteins, of a pore, resulting in lysis of the affected cell. C3b also plays a role as an opsonine and facilitates phagocytosis by macrophages.

### 1) Materials and Methods

The activation of the classical complement pathway by the anti-DGDG antibodies of example 1 was measured according to the “TH50” determination protocol (TH50 being the time necessary to observe 50% hemolysis, Abbal et al., Complement Inflammation, 1991, 8, 92-103) with determination of the presence of the hemolytic C4 protein (Gaither et al., J. Immunol., 1974, 113, 574-583). Briefly, sheep erythrocytes are sensitized with an anti-erythrocyte antibody so as to form a sensitized particle. A solution of human serum (NHS) is then added to the solution containing this sensitized particle. The NHS contains all the complement proteins, including the C1q complex necessary for initiation of the classical complement pathway. On contact with the sensitized erythrocyte, C1q initiates the complement cascade, thereby leading to lysis of the red blood cells. This lysis, and more particularly the release of the hemoglobin which results therefrom, is measured over time by spectrometry, and the curve inflexion point constitutes the time necessary for lysis of 50% of the red blood cells (TH50). This measurement constitutes the reference TH50.

Freshly lyzed extracellular parasites (8×10⁶ per condition) were incubated in the presence of several dilutions of the anti-DGDG rabbit serum (1/1, 1/2, 1/5, 1/10, 1/20, 1/50 and 1/100) and also with pre-immune serum. The parasites were then rinsed in PBS and then incubated for 1 hr at 37° C. with NHS seronegative for toxoplasmosis. During this step, the complement proteins may partly be consumed by the serum-antigen complex. The NHS-serum-antigen solution is then mixed with the solution containing the sensitized red blood cells. The TH50 measurement is then carried out. The ability of a serum to activate the complement results in an increase in the reference TH50, linked to the decrease in the number of erythrocytes lyzed. It can therefore be quantified relative to the control. The reference TH50 was measured in a sensitized red blood cell-NHS-PBS mixture. In addition, due to the fact that some pathogens are capable of activating complement (alternative pathway and lectin pathway) merely by their presence, a control measurement for the TH50 was carried out on extracellular parasites without serum pretreatment with NHS.

### 2) Results

The TH50 measurements (FIG. 14) show that the pretreatment of the toxoplasms with the anti-DGDG rabbit serum (dilutions 1/1 to 1/10) activates the classical complement pathway significantly. This activation reaches a maximum at the 1/2 dilution (42% remaining complement activity only, compared with the 100% of the reference with parasites PBS). It is at this dilution that the stoichiometry conditions appear to be most favorable to the reaction. The pretreatment with the anti-DGDG serum diluted to 1/10, to 1/5 or to 1/1 results in a marked decrease in the remaining complement activity, but which is proportional to the dilution, compared with the PBS control. It is interesting to note that the parasites alone, incubated in the presence of NHS and of sensitized red blood cells, induce a decrease in this remaining complement activity, compared with the PBS control (77% activity only). These results suggest that the toxoplasma might activate the complement cascade independently of the presence of specific antibodies; it might therefore activate the alternative pathway or the lectin pathway.

1. A method of preventing or treating an infection by an apicomplexan parasite comprising administering to a mammal an effective dose of a digalactolipid antigen, expressed at the outer surface of the plasma membrane of apicomplexan parasites at all the parasitic stages, or an antibody directed against said antigen, or a functional fragment of said antibody.

2. The method of claim 1, wherein said antigen comprises digalactose 6-O-(α-D-galactopyranosyl)-β-D-galactopyranose covalently associated with a lipid.

3. The method of claim 1, wherein said antigen is a digalactosyldiradylglyceride of formula I: 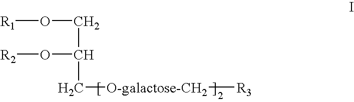
wherein R₁ and R₂ each represent, independently of one another, an alkyl or an acyl containing 10 to 25 carbon atoms, and R₃ represents a hydroxyl or sulfonate group.

4. The method of claim 3, wherein R₁ and R₂ each represent an acyl.

5. The method of claim 3, wherein R₁ and/or R₂ represent the hydrocarbon-based chain of linolenic acid.

6. The method of claim 3, wherein said antigen of formula I is 1,2-diacyl-(α-D-galactopyranosyl-(1′→6′)-β-D-galactopyranosyl-(1′→3))-sn-glycerol.

7. The method of claim 1, wherein said antibody or antibody fragment is monoclonal antibodies, polyclonal antibodies, or chimeric antibodies, or the Fab, Fv and scFv fragments of said antibodies.

8. The method of claim 1, wherein said antigen, antibody or antibody fragment is coupled to a label.

9. The method of claim 1, wherein said antigen, antibody or antibody fragment is immobilized on a support.

10. The method of claim 1, wherein said parasite is a pathogen of the Plasmodium, Babesia, Toxoplasma, Neospora, Cryptosporidium, Theileria, Sarcocystis, Eimeria or Gregarina genus.

11. The method of claim 10, wherein said pathogen is a human pathogen selected from the group consisting of Plasmodium faciparum, Plasmodium vivax, Plasmodium ovale, Plasmodium malariae, Babesia microti, Toxoplasma gondii and Cryptosporidium hominis.

12. The method of claim 10, wherein said pathogen is an animal pathogen selected from the group consisting of Babesia divergens, Babesia bigemina, Babesia major, Babesia bovis, Babesia canis, Babesia gibsoni, Babesia equi, Babesia caballi, Neospora caninum, Cryptosporidium parvum, Theileria annulata, Theileria parva, Sarcocystis neurona, Eimeria tenella and Gregarina niphandrodes.

13. An in vitro method for detecting an infection by an apicomplexan parasite comprising:
(a) obtaining a biological sample from an individual who may be infected by said parasite;
(b) bringing said biological sample into contact with at least one antigen, one antibody or one antibody fragment of claim 20; and
(c) revealing the antigen-antibody complexes formed in (b).

14. The method of claim 13, wherein step (b) comprises bringing said biological sample into contact successively with at least a first capture antibody, or a fragment thereof directed against the digalactolipid antigen and at least a second antibody, or a fragment thereof, specific for the parasite to be detected, wherein the second antibody is different than the first antibody.

15. The method of claim 14, wherein the second antibody is an antibody specific for the genus or for the species of the parasite to be detected.

16. The method of claim 13, further comprising, prior to or concomitantly with step (b), permeabilizing a membrane.

17. The method of claim 13, wherein the method is an ELISA immunocapture method.

18. The method of claim 13, wherein the method is an immunochromatography method.

19. A diagnostic reagent comprising a digalactolipid antigen, expressed at the outer surface of the plasma membrane of apicomplexan parasites at all the parasitic stages, or an antibody directed against said antigen, or a functional fragment of said antibody.

20. An isolated digalactolipid antigen, expressed at the outer surface of the plasma membrane of apicomplexan parasites at all the parasitic stages, or an isolated antibody directed against said antigen, or a functional fragment of said antibody.

21. An immunogenic or vaccine composition comprising the antigen of claim 20.

22. The composition of claim 21, wherein the other antigen of interest is an antigen specific for an apicomplexan parasite.

23. The antibody of claim 20, wherein said antibody is a monoclonal antibody or functional fragment of said antibody.

24. A method of diagnosing an infection by an apicomplexan parasite comprising use of the antigen, or the antibody, or the functional fragment of said antibody of claim 20.
